(19) United States
(12) Patent Application Publication
(10) Pub. No.: US 2026/0284256 A1
(43) Pub. Date: Sep. 24, 2026

(54) SYNTHETIC GRAFT, METHOD OF MANUFACTURE AND USES THEREOF

(71) Applicant: University of Connecticut, Farmington, CT (US)

(72) Inventors: Cato T. Laurencin, Farmington, CT (US); Lakshmi S. Nair, New Britain, CT (US); Chinedu C. Ude, Farmington, CT (US); James H. Chapman, Orange, CT (US)

(21) Appl. No.: 19/476,067

(22) PCT Filed: Apr. 17, 2024
(86) PCT No.: PCT/US2024/025029

§ 371 Date: Oct. 16, 2025

Related U.S. Application Data
(60) Provisional application No. 63/459,763, filed on Apr. 17, 2023.

(51) Int. Cl. A61L 27/18; A61L 27/58

(52) U.S. Cl. CPC A61L 27/18; A61L 27/58; A61L 2430/10

(57) ABSTRACT

Disclosed herein is a scaffold for use in a body of a living being, the scaffold comprising a biodegradable portion; and a non-degradable portion; where the biodegradable portion form the major portion of the scaffold; and where the biodegradable portion and the non-degradable portion both comprise biocompalible polymers. Disclosed herein too is a method of manufacturing a scaffold for tissue regeneration, the method comprising braiding a first fiber with a second fiber to form a braid; where the first fiber comprises a biodegradable polymer and where the second fiber comprises a non-degradable polymer; where the first fiber is present in an amount of greater than 50 wt %, based on a total weight of the scaffold.

### CROSS-REFERENCE TO RELATED APPLICATIONS

This disclosure claims priority to U.S. Provisional Application No. 63/459,763, filed on Apr. 17, 2024, the entire contents of which are incorporated herein in their entirety.

### GOVERNMENT SUPPORT

This invention was made with government support under AR079114 awarded by the National Institutes of Health, and 1332329 awarded by the National Science Foundation. The government has certain rights in the invention.

### FIELD OF THE DISCLOSURE

The invention disclosed herein relates to a synthetic graft, method of manufacture and to uses thereof, and in particular to a biomimetic graft for use in soft tissue and ligament regeneration.

### BACKGROUND

The anterior cruciate ligament (ACL) is an important support structure for the knee joint and is unfortunately often damaged by athletes. ACL injuries are common, especially among athletes involved in sports that require sudden stops, changes in direction, or jumping. These injuries often occur due to twisting or hyperextension of the knee. Symptoms of an ACL injury can include a popping sound or sensation at the time of injury, severe pain, swelling, instability or a feeling of the knee giving way, and difficulty bearing weight on the affected leg.

ACL injuries are typically diagnosed through a combination of physical examination, imaging tests (such as MRI), and assessment of the patient's symptoms and medical history. Treatment for ACL injuries depends on various factors, including the severity of the injury, the patient's activity level, and the presence of other knee injuries. Options may include rest, physical therapy, bracing, and in some cases, surgery to reconstruct the torn ligament. Recovery from an ACL injury can be a lengthy process, often involving rehabilitation exercises to strengthen the knee muscles and regain range of motion. Return to sports or other high-impact activities may take several months and should be guided by a healthcare professional.

ACL reconstruction involves replacing the damaged ACL with natural tissue (such as the patient's own tendon or a donor tendon) via surgery. Synthetic grafts made from materials such as polyester, polyethylene terephthalate (PET), or polypropylene have been used in lieu of natural tissue.

These synthetic grafts unfortunately have drawbacks one of which is biocompatibility. Because synthetic tissues are not as biocompatible as natural tissue, this increases the risk of inflammation, rejection, or infection. The lack of compatibility of synthetic grafts causes the healing process to differ from that of natural tissue, potentially affecting the long-term stability (life-cycle) and function of the reconstructed ACL. Because of these compatibility and life-cycle issues, a fully synthetic graft has yet to gain FDA approval for ACL reconstruction.

There is therefore an immediate need for a novel synthetic graft that offers additional structural support without some of the aforementioned drawbacks.

### SUMMARY

Disclosed herein is a scaffold for use in a body of a living being, the scaffold comprising a biodegradable portion; and a non-degradable portion; where the biodegradable portion form the major portion of the scaffold; and where the biodegradable portion and the non-degradable portion both comprise biocompatible polymers.

Disclosed herein too is a method of using a scaffold for regenerating tissue, the method comprising disposing a scaffold into two bones located across a joint in a body of a living being;
where the scaffold comprises a biodegradable portion; and a non-degradable portion; where the biodegradable portion form the major portion of the scaffold; and where the biodegradable portion and the non-degradable portion both comprise biocompatible polymers; and where a first end of the scaffold is affixed to a first bone and a second end of the scaffold is affixed to a second bone; where the first bone and the second bone are located on opposite sides of the joint; and degrading the biodegradable portion of the scaffold; and depositing cells into the scaffold to facilitate tissue regeneration.

Disclosed herein too is a method of manufacturing a scaffold for tissue regeneration, the method comprising braiding a first fiber with a second fiber to form a braid; where the first fiber comprises a biodegradable polymer and where the second fiber comprises a non-degradable polymer; where the first fiber is present in an amount of greater than 50 wt %, based on a total weight of the scaffold.

### BRIEF DESCRIPTION OF THE FIGURES

FIG. 1 depicts a tunnel that is drilled in each of the femur and the tibia with the dotted lines showing the composite ligament that is used for eventual tissue regeneration.

### DETAILED DESCRIPTION

The term “scaffold” as used herein, may be interchangeable with similar terms such as “implant”, “stent,” “substrate” and, in some cases “prosthesis.” In some embodiments, the scaffold is tubular and suited for therapy of an opening (in normally occurring tissue inside the body of a living being) or other anomaly, such as a perforation.

The scaffold may be partially tubular, C-shaped, a stripe or of another shape such as an irregular or contoured shape. In short, the scaffold may exhibit any geometry deemed suitable by a medical practitioner in order to repair a damaged section of an organ present in the body of a living being. In some embodiments, the scaffold is configured for developing new or original tissue that was omitted,

The term “living being” as used herein applies to any species of multicellular organism that has a capacity for one or more of homeostasis, organization, metabolism, growth, adaptation, response to stimuli, and reproduction. The term “living being” encompasses vertebrates and invertebrates. It encompasses mammals, fish, birds, reptiles and amphibians. Preferred examples of living beings that can use the composite ligaments include animals commonly regarded as pets such as dogs, cats, horses, bulls, cows, goats, sheep, elephants, monkeys, lions, and the like.

The term “autologous cells” refer to cells that are obtained from and used in the same individual. In other words, they are cells that are harvested from a person's own body for therapeutic or medical purposes, often for the purpose of transplantation or regenerative therapies. Autologous cells are advantageous in that they reduce the risk of rejection or immune reactions, as the cells are recognized as “self” by the immune system. Autologous stem cell transplantation (also called autogenous, autogenic, or autogenic stem cell transplantation) includes transplantation in which stem cells (undifferentiated cells from which other cell types develop) are removed from a person, stored, and later given back to that same person.

Disclosed herein is a composite ligament that may be used as a replacement for a damaged anterior cruciate ligament (ACL), medial collateral ligament (MCL), posterior cruciate ligament (PCL), lateral collateral ligament (LCL), or a combination thereof. In a preferred embodiment, the composite ligament is used to facilitate replacement and repair for a damaged ACL. The composite ligament is generated in-situ in the body of a living being and comprises a biodegradable polymer and a non-biodegradable polymer. Both the biodegradable polymer and the non-biodegradable polymer are biocompatible and do not suffer rejection when inserted in the body of a living being.

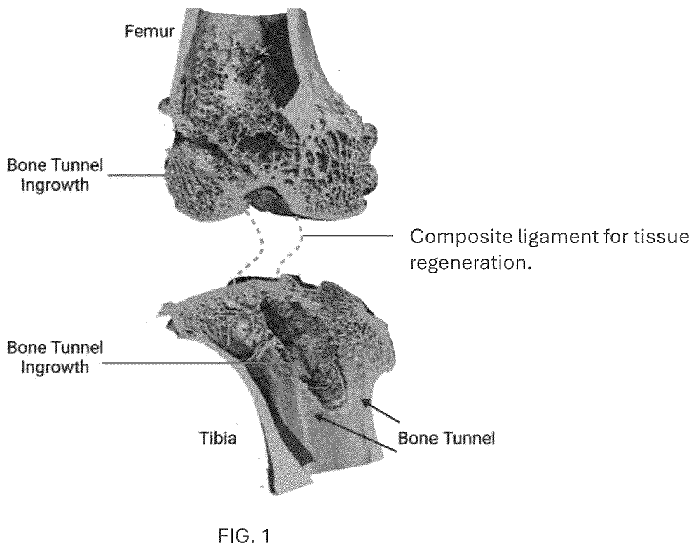

Disclosed herein too is a method of manufacturing the composite ligament as well as a method of using the composite ligament in the body of a living being. The method comprises using the composite ligament as a scaffold across a joint that has opposing bones (e.g., such as in the knee, where the opposing bones are the femur and the tibia or the fibula, or the elbow, where the opposing bones are the humerus and either the radius or ulna). The opposing bones are referred to herein as the first bone and the second bone. As seen in the FIG. 1, a first tunnel is formed in the femur (the first bone) while a second tunnel is formed the tibia (the second bone). The composite ligament is placed with opposing ends located in the tunnel of each bone. For example, a first end of the composite ligament is placed in the first tunnel in the femur (the first bone) and the opposite end (the second end) of the composite ligament is placed in the second tunnel in the tibia (the second bone). Over time, tissue regeneration occurs in the intra-articular region of the composite ligament between the tunnels (as well as in the tunnels). The tissue regeneration will eventually lead to the formation of a new ligament to replace the damaged ligament. During the tissue regeneration, the biodegradable material undergoes degradation, while the non-degradable material provides the scaffold with mechanical strength and integrity to stabilize the scaffold.

The composite ligament comprises a novel composite synthetic (polymer) graft with both biodegradable and non-degradable polymers. The biodegradable portion degrades over time, while the non-degradable portion provides mechanical support to the replacement ligament as it regenerates. In an embodiment, the composite ligament is a scaffold that comprises one or more interwoven or braided filaments that comprise the biodegradable polymer and the non-degradable polymer. This design includes a decreased percent of non-degradable polymer relative to the biodegradable polymer content. This permits rapid biodegradation of the scaffold, while still providing structural support through the non-degradable polymer. The non-degradable filaments of the scaffold therefore provide structural support, while the biodegradable filaments facilitate tissue infiltration.

As noted above, the biodegradable portion of the scaffold undergoes degradation over time while the scaffold is located in the body of a living being. During this period, tissue infiltration occurs in the scaffold to facilitate regeneration of the ligament.

Tissue infiltration may occur via a variety of different cells in the body. The cells are broadly described as therapeutic cells. Therapeutic cells generally refer to biological cellular materials useful for initiating therapy for a patient. Examples include autologous cells or autologous adipose derived mesenchymal stromal cells (Ad-MSC) as well as others. Ad-MSCs possess a high regenerative potential and immunomodulatory activities. Ad-MSCs have great potential in stem cell-based therapies and wound healing as well as in orthopedic, cardiovascular, or autoimmune diseases. Generally, mesenchymal stem cells (MSCs) are multipotent stem cells that can differentiate into a variety of cell types, including bone cells (osteoblasts), cartilage cells (chondrocytes), muscle cells (myocytes) and fat cells that give rise to marrow adipose tissue (adipocytes). In a preferred embodiment, tissue infiltration by mesenchymal stem cells is preferred because these cells can differentiate themselves based on phenotype of the local environment in the body region that the scaffold is used. The phenotype of MSCs refers to their characteristic properties and behavior, which can vary depending on their source, environment, and specific conditions.

The biodegradable polymer is selected from the group consisting of poly L-lactic acid (PLLA), polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D, L-lactic acid-p-dioxanone-polyethylene glycol block copolymer (PLA-DX-PEG), or the like, or a combination thereof.

A preferred biodegradable polymer is poly L-lactic acid (PLLA).

The biodegradable portion of the scaffold is the major portion of the scaffold and is present in an amount of greater than 50 weight percent (wt %), preferably greater than 60 wt %, preferably greater than 70 wt %, preferably greater than 80 wt %, and more preferably greater than 90 wt %, based on a total weight of the scaffold. In an embodiment, the biodegradable portion of the scaffold may be about 95 wt %, based on a total weight of the scaffold.

The non-degradable polymer provides mechanical strength to the scaffold during tissue regeneration. The non-degradable polymer is preferably one that is biocompatible and does not decompose to form toxic byproducts. Even if the non-degradable polymer is not perfectly biocompatible it may be coated with a polymer that is biocompatible such as a polysiloxane, a polyfluoroethylene, a polyolefin, or the like, or a combination thereof.

The non-degradable polymer be may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination thereof.

The organic polymer has a number average molecular weights greater than 10,000 grams per mole, preferably greater than 20,000 g/mole and more preferably greater than 50,000 g/mole. In an embodiment, the organic polymer has a number average molecular weight of up to 500,000 g/mole.

Examples of thermoplastic polymers include a polyacrylic, a polycarbonate, a polyalkyd, a polystyrene, a polyolefin, a polyester, a polyamide, a poly aramid, a poly amideimide, a polyarylate, a polyurethane, an epoxy, a phenolic, a polysiloxane, a polyarylsulfone, a polyethersulfone, a polyphenylene sulfide, a polysulfone, a polyimide, a poly etherimide, a polytetrafluoroethylene, a polyetherketone, a polyether ether ketone, a polyether ketone ketone, a polybenzoxazole, a polyoxadiazole, a polybenzothiazole, a poly benzothiazinophenothiazine, a polypyrazinoquinoxaline, a polypyromellitimide, a poly guinoxaline, a poly benzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a poly sulfonamide, a polyurea, a polyphosphazene, a polysilazane, a polyolefin, or the like, or a combination thereof.

As noted above, the aforementioned polymers are preferably biocompatible and when the polymer is not biocompatible, it may be coated with a biocompatible polymer to reduce rejection by the body.

Preferred polymers for use as non-degradable polymers include poly olefins (e.g., polyethylene, polypropylene, copolymers of polyethylene, or a combination thereof), poly siloxane (e.g., polydimethylsiloxane), polyfluoroethylene (e.g., polytetrafluoroethylene, perfluoroelastomers, fluorinated ethylene propylene, or a combination thereof), or polyester (e.g., polyethylene terephthalate (PET), polybutylene terephthalate, or a combination thereof). A preferred polymer for use as a non-degradable polymer includes polyethylene terephthalate (PET).

The non-degradable portion of the scaffold is the minor portion of the scaffold and is present in an amount of less than 25 weight percent (wt %), preferably less than 15 wt %, preferably less than 12 wt %, preferably less than 10 wt %, and more preferably less than 5 wt %, based on a total weight of the scaffold. In an embodiment, the biodegradable portion of the scaffold may be about 3 to 8 wt %, based on a total weight of the scaffold.

The scaffold is prepared by weaving or braiding fibers of the biodegradable polymer and the non-degradable polymer. Braiding is preferred. Braiding of fibers involves intertwining multiple strands of material to create a stronger, more durable structure.

In another embodiment, the scaffold may be manufactured by extruding the biodegradable and the non-degradable polymer to form a cylinder or a tube. The cylinder or the tube may then be used as a scaffold for tissue regeneration. In an embodiment, opposing ends of the scaffold are designed to be fitted into tunnels in opposing bones across a joint (such as a knee or elbow) in a body of a living being.

In one embodiment, in one manner of using the scaffold, surgery is performed on a patient that has a damaged ligament such as, for example, a torn ACL. During surgery, tunnels are drilled into the femur and tibia and the opposing ends of the scaffold are inserted into the tunnels in the tibia and femur. Over time, it is observed that the biodegradable tissue degrades while at the same time tissue regeneration occurs in the scaffold. Tissue regeneration in the tunnel occurs for a period that ranges from 1 day to 1 year, preferably 10 days to 9 months, and more preferably 2 to 6 months. Tissue regeneration between the opposing ends of the scaffold (in the intra-articular region) is highly desirable. The intra-articular region lies between the opposing ends of the scaffold that are typically placed in the tunnels in the tibia and the femur.

In one embodiment, the scaffold may be used to regenerate tissue in situ-i.e., it is placed in lieu of a damaged ligament and gradually regenerates tissue to replace the damaged tissue. In another embodiment, the scaffold after a period of 6 to 9 months may be removed from the tunnels and used to replace a damaged or torn ligament elsewhere in the body.

In yet another embodiment, the tunnels may be used to regenerate tissues that can used upon demand. In other words, if an athlete has a high probability of damage to one of his/her ligaments, then tunnels can be proactively created in the bones to generate ligaments that may be used in the event of a future accident (that damages the (ACL), medial collateral ligament (MCL), posterior cruciate ligament (PCL), lateral collateral ligament (LCL), or a combination thereof). In other words, the living being may be used as a repository for some body parts that may be used as replacement tissue in the event of a future accident.

Certain embodiments of this disclosure are disclosed in U.S. Pat. No. 8,758,437B2, the entire contents of which are incorporated herein by reference in their entirety.

The composite ligament disclosed herein is exemplified by the following non-limiting example.

### EXAMPLE

This example was conducted to demonstrate the manufacturing of a scaffold and its use in tissue regeneration. Biodegradable poly-L-lactic acid (PLLA) was combined with non-biodegradable polyethylene terephthalate (PET) to form the composite ligament. The composite ligament was manufactured by braiding.

The composite ligament (also sometimes referred to as a “graft”) was implanted in the left knee of 12 New Zealand white rabbits for an extended period of 6 months with ensuing biomechanical and histomorphometric analysis.

Gross image revealed that 11/12 grafts were intact at 6 months, however their volume shrunk compared to the original graft at time of implantation. There was evidence of bone tunnel osteogenesis measured by micro computerized tomography (μCT) scans.

Biomechanical testing revealed diminished strength compared to the prior data at 3-month in vivo time point. The histology and scanning electron microscopy demonstrated evidence of tissue regeneration and possible replacement of the biodegradable component PLLA. Specifically, the Goldner trichrome and hematoxylin-eosin staining demonstrated infiltration of new tissue into the graft within the joint space.

These results indicate that a semi-biodegradable polymer graft (the composite ligament) offers additional early structural support for a replacement for damaged ligaments. Using exogenous growth factors and tissue support may offer enhanced cellular recruitment and proliferation in the synovial environment as an area of future work.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. A scaffold for use in a body of a living being, the scaffold comprising:
a biodegradable portion; and
a non-degradable portion; where the biodegradable portion form the major portion of the scaffold; and where the biodegradable portion and the non-degradable portion both comprise biocompatible polymers.

2. The scaffold of claim 1, where the scaffold is in the form of a braid; where the braid is manufactured by braiding fibers that comprise the biodegradable portion with fibers that comprise the non-degradable portion.

3. The scaffold of claim 1, where the scaffold is in the form of a cylinder or a tube.

4. The scaffold of claim 1, where the biodegradable portion is selected from the group consisting of poly L-lactic acid, polylactic-glycolic acid, poly-caprolactone, copolymers of polylactic-glycolic acid and poly-caprolactone, polyhydroxy-butyrate-valerate, polyorthoester, polyethylene oxide-butylene terephthalate, poly-D,L-lactic acid-p-dioxanone-polyethylene glycol block copolymer, or a combination thereof.

5. The scaffold of claim 1, where the biodegradable portion comprises poly L-lactic acid.

6. The scaffold of claim 1, where the non-degradable portion comprises at least one of a polyolefin, a polysiloxane, a polyfluoroethylene, a polyester or a combination thereof.

7. The scaffold of claim 6, where the polyester is polyethylene terephthalate.

8. The scaffold of claim 1, where the biodegradable portion is present in an amount of greater than 80 wt%, based on a total weight of the scaffold.

9. The scaffold of claim 1, where the non-degradable portion is present in an amount of less than 20 wt %, based on a total weight of the scaffold.

10. The scaffold of claim 1, where opposing ends of the scaffold are designed to be fitted into tunnels in opposing bones across a joint in a body of a living being.

11. A method of using a scaffold for regenerating tissue, the method comprising:
disposing a scaffold into two bones located across a joint in a body of a living being; where the scaffold comprises:
a biodegradable portion; and
a non-degradable portion; where the biodegradable portion form the major portion of the scaffold; and where the biodegradable portion and the non-degradable portion both comprise biocompatible polymers; and
where a first end of the scaffold is affixed to a first bone and a second end of the scaffold is affixed to a second bone; where the first bone and the second bone are located on opposite sides of the joint; and
degrading the biodegradable portion of the scaffold; and
depositing cells into the scaffold to facilitate tissue regeneration.

12. The method of claim 11, where the first end of the scaffold is affixed to the first bone via a tunnel in the first bone.

13. The method of claim 11, where the second end of the scaffold is affixed to the second bone via a tunnel in the second bone.

14. The method of claim 11, where the cells are therapeutic cells.

15. The method of claim 14, where the therapeutic cells are mesenchymal stem cells.

16. The method of claim 11, where the first bone is a femur and the second bone is a tibia.

17. The method of claim 11, where the scaffold is disposed in the two bones prior to tissue damage.

18. A method of manufacturing a scaffold for tissue regeneration, the method comprising:
braiding a first fiber with a second fiber to form a braid; where the first fiber comprises a biodegradable polymer and where the second fiber comprises a non-degradable polymer; where the first fiber is present in an amount of greater than 50 w t%, based on a total weight of the scaffold.

19. The method of claim 18, where the first fiber is present in an amount of greater than 80 wt %, based on a total weight of the scaffold.

20. The method of claim 18, where the second fiber is present in an amount of less than 20 wt %, based on a total weight of the scaffold.
